(19) United States
(12) Patent Application Publication
(10) Pub. No.: US 2009/0076329 A1
(43) Pub. Date: Mar. 19, 2009

(54) Disposable Stereoscopic Endoscope System

(76) Inventors: Wei Su, Sunnyvale, CA (US); Gary Zhang, Sunnyvale, CA (US)

Correspondence Address: Wei Su, 1567 Samedra St, Sunnyvale, CA, 94087, US

(21) Appl. No.: 12/212,625

(22) Filed: Sep. 17, 2008

Related U.S. Application Data
(60) Provisional application No. 60/993,858, filed on Sep. 17, 2007.

(51) Int. Cl. A61B 1/06; A61B 1/005

(52) U.S. Cl. 600/134; 600/166

(57) ABSTRACT

A disposable simultaneous stereo endoscope system is disclosed. The disposable endoscope does not include image relay. Instead, two electronic imaging sensors and solid illumination lighting are arranged inside the endoscope. A demultiplexing beam splitter is used for splitting the two imaging light beams to the two imaging devices. A wedged multi-facet illumination window is used to create an illumination field that is larger than the field of view of the imaging optics. An electrically conductive heat sink is engaged for dissipating the heat generated by the solid light source and also for shielding end side of the endoscope. The disposable endoscope is shielded from electromagnetic interferences. A repeater unit is used to electrically connect the disposable endoscope with a remote receiver and to increase the data transfer rate. An electrical isolation means is provided between the endoscope and an image processing and power conditioning unit to protect the endoscope against electric shock.

### CROSS REFERENCE TO RELATED APPLICATIONS

This application claims the benefit of provisional patent application Ser. No 60/993,858, filed Sep. 17, 2007 by the present inventors.

This application is related to a co-pending U.S. patent application entitled “Compact Stereoscopic Endoscopes” which is incorporated herein by reference.

### US Patents

U.S. Pat. No. 5,222,477
U.S. Pat. No. 6,720,988
U.S. Pat. No. 6,976,956
U.S. Pat. No. 7,230,756

### FIELD OF THE INVENTION

The present invention generally relates to endoscopes and in particular, to the design of a stereoscopic endoscopes system for medical procedures and industrial applications.

### BACKGROUND OF THE INVENTION

An endoscope is an imaging device capable of transmitting image from its tip (or distal end) through its long, slim shank to an imaging sensor so that a live image can be displayed on an electronic monitor at a distance. An endoscopic system often consists of four major parts: a miniature imaging optics, image transmission optics, electronic imaging sensor and display. Endoscopes are generally required to be of certain length since they are usually operated outside patients' body and the distal imaging optics collects and conveys the images of inner anatomy to the outside. Since the imaging electronics is too bulky to be placed close to the imaging optics, the image transmission optics becomes a critical component, which also defines the basic architectural design of any endoscope. Before the coherent fiber optic cable was invented and used to relay an optical image over the full length of the cable, miniature relay lens system was generally used to transmit the optical image. Due to the fixed positions of the relay lenses, endoscopes can only be made with a rigid shank, limiting its potential applications. The flexibility of the fiber optical cable has made flexible endoscopes a reality. However, the inherent low resolution and high optical attenuation resulting from limitation of the optical fiber has limited the performance of endoscopes based on fiber cables. The endoscopes with similar optical design and construction are also used widely in industrial supplications for inspecting defect or objects in places difficult to be accessed from a small opening.

The endoscopes on market today are mostly designed for multiple uses in medical procedures, requiring various levels of disinfection and rigorous reprocessing after each use. The shortcomings of repeated use include the risk of potential cross contamination, high cost of a sustainable design, tear and wear in function, and increased personnel cost for maintenance. Given these issues, a disposable endoscope apparently has its clear advantage. However, the cost of optics, especially that of the image transmission optics and electronic imaging sensors have so far kept the cost high even for endoscopes designed as disposables (U.S. Pat. No. 7,230,756 B2). Besides, the size of the imaging electronics prevents its placement close to the imaging optics and as a consequence the design still relies on traditional relay optics for image transmission.

Today most endoscopes are not designed and manufactured to generate stereoscopic images. Prior art stereo endoscope not only requires dual apertures (pupils) for generating two independent imaging channels, but also need a relative large space to transmit the two image through two spatially non-overlapping optical paths. Such a spatial division based configuration also result in a larger dimension of the endoscope, and hence is unlikely to meet the requirement of minimally invasive surgeries.

There are two traditional architectural designs for stereo endoscopes. One typical design uses the relay optical system to transmit the optical image from the distal end to two imaging sensors located at the other end (U.S. Pat. No. 6,976,956, and U.S. Pat. No. 6,720,988). Due to the higher cost of relay optics for dual channel stereo endoscopes, these types of endoscopes are very expensive and not applicable for disposable applications. The second design places the electronic imaging sensors directly behind the imaging optics and removes the expensive relay optics from the endoscope (U.S. Pat. No. 5,222,477). However, the design proposed in patent U.S. Pat. No. 5,222,477 is based on sequential stereo imaging, in which the images from the two independent channels are captured one at a time. When the captured video images are displayed on a stereoscopic monitor, a slight motion of the targeted object or endoscope itself would result in false stereoscopic acuity for the observer, which will result in the impression that the object is moving in-and-out of the monitor screen.

In addition to issues related to the imaging and relay optics, from a system level perspective, traditional endoscopes also have limitations in optical illumination, EMI shielding, electrical isolation, and other safety related issues. For example, traditionally, the illumination light is guided from a high intensity lamp to the imaging site by an optical fiber cable. The small size and numerical aperture of the fiber cable result in huge light loss during its coupling and transmission. As a result, high power short arc lamps, which often have short life time, have to be used to compensate for the low efficiency of illumination light transmission.

Practically, all of endoscopes today use electronic imaging system to transfer the optical image captured by the endoscope to an electronic display monitor. The image capturing device is attached to the endoscope at its proximal end and located at a distance from the patient. Because of this, only simple Basic Insulation is required for the electronics when it is further isolated electrically from the distal end of the endoscope. Even when Reinforced Insulation is required for the imaging electronics, it is rather simple to implement due to lax constraint on the available space in the area. It is much more difficult to implement the required electrical insulation when the miniature electronics is implemented into the distal end of the endoscope. So far, very few attentions have been paid to the electrical safety issues.

### OBJECTS AND SUMMARY OF THE INVENTION

In this invention, the word “disposable endoscope” means an endoscope that is relatively compact and low cost so that one can afford to use it for only a limited number of times before throwing it away.

The present invention discloses a disposable simultaneous stereoscopic endoscope system that is compact and low cost. In addition, a number of system level issues have been addressed to make the system suitable for meeting the safety regulatory requirement.

One object of this invention is to design a disposable unit of the endoscope that contains both the imaging and the illumination sub-systems so that the disposable unit only needs to be connected to the rest of the system electrically. The imaging sub-system uses two spatially separated sub-apertures for defining two stereo imaging channels and a demultiplexing beam splitter for splitting the two light beams to two imaging devices to make the sub-system compact. The illumination sub-system uses more efficient and compact solid state light sources such as high brightness white LEDs (light emitting diodes) or LDs (laser diodes) or SLEDs (superluminescent light emitting diodes) to replace traditional high intensity illumination lamps. Meanwhile, light from the solid light source is coupled into optical fibers based ring illumination path, and a wedged multi-facet illumination window is used to modify the illumination light ray so that the illumination field is slightly larger than the field of view of the imaging sub-system for the desired imaging space of the endoscope.

Another object of this invention is to electrically connect the disposable imaging and lighting endoscope unit with a repeater unit containing a power conditioning/distribution module and an input/output signal conditioning and control module so that high data transfer rate can be maintained over a relatively long distance.

Another object is to electrically isolate the disposable endoscope and its electronics from the patient to satisfy the electric safety requirement of medical device and to provide protection means for the patient against electric shocks in the case of device failure.

Another objective is to reduce the exposure of EMI (electromagnetic interference) to the patient from the working electronics inside the distal end of the endoscope, and meanwhile provide EMI shielding to the sensitive electronics in the endoscope from the environment.

Another objective is to use proper thermal sink at the distal end of the endoscope, to dissipate heat generated by the solid lighting source and electronics for prolong operation and to integrate the heat sink with the EMI shielding.

Another objective is to install an orientation detection sensor to the endoscope, so that the displayed stereo video images could be adjusted based on the orientation of the endoscope.

Still another objective is to add writable memory chip to the disposable part of the endoscope so that information about the disposable device and its usage could be recorded and tracked; the initial setup of the disposable device to the base station could be automated and the device could cease to function when the limit for usage is reached. Imaging parameters may also be stored in this memory for later retrieval to correct optical distortions and electronic defects.

Additional objects, features and advantages of the various aspects of the present invention will become apparent from the following description of its preferred embodiment, which description should be taken in conjunction with the accompanying drawings.

### BRIEF DESCRIPTION OF THE DRAWINGS

FIG. 1 shows a section view of the disposable imaging and lighting unit of the presently disclosed stereoscopic endoscope.

FIG. 2 shows the illumination field and the imaging field, where the illumination field is larger than the imaging field, but still covers the field of view of the endoscope.

FIG. 3 shows the electrical connection between the disposable imaging and lighting unit and a unit containing a power conditioning/distribution module and an input/output conditioning module.

FIG. 4 shows a Basic Protective Earth Insulation design of the presently disclosed simultaneous stereo endoscope system.

FIG. 5 shows a Double Insulation design of the presently disclosed simultaneous stereo endoscope system.

FIG. 6(a) shows one embodiment of the lighting unit in which the solid light source is directly connected to the fiber cable for illumination light delivery.

FIG. 6(b) shows another embodiment of the lighting unit in which light from the solid light source is coupled to the fiber cable via a condensing lens.

FIG. 7 shows another embodiment of the lighting unit in which an optical beam combiner is used to combine light beams from at least two light sources with different wavelengths, to form an equivalent light source of multiple wavelengths, and the light beam is coupled into the fiber cable through a condensing optical lens and/or light homogenization device.

### DETAILED DESCRIPTION OF THE PREFERRED EMBODIMENT

A novel simultaneous stereoscopic endoscope system based on solid state lighting is proposed. One unique feature of the present invention is the compact design of stereoscopic imaging and lighting within a disposable housing such that only electrical signal connection is required between the disposable part and the rest of the endoscope system for transmitting power, imaging and control signals. In particular, a miniature low cost digital imaging unit is placed directly at the distal end of the endoscope, which eliminates the need for a relay optical system. The new optical design makes it possible to miniaturize the imaging unit to the size similar to traditional optical endoscopes.

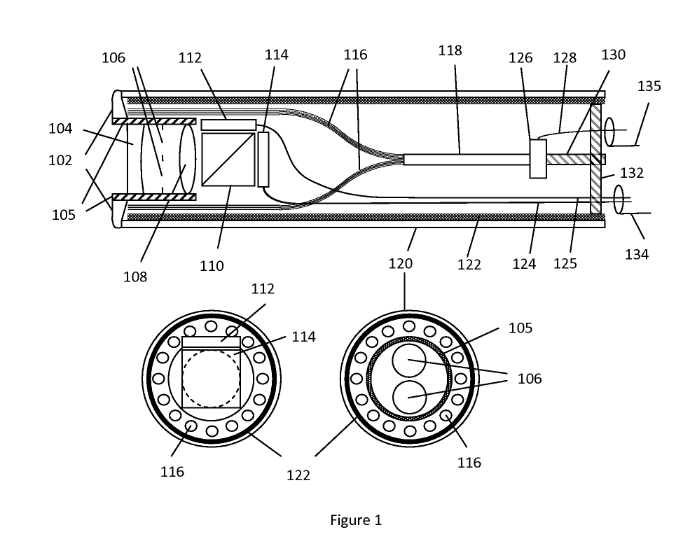

FIG. 1 shows a section view of the disposable imaging and lighting unit of the presently disclosed stereoscopic endoscope. In terms of imaging optics, two optical modules such as the lenses 104 and 108 are used to form two images of an object onto two electronic imaging sensors 112 and 114. The optical aperture 106 is placed between two optical modules 104 and 108 and it is composed of two symmetrically located sub-apertures that offer optical multiplexing characteristics. The beam splitter cube 110 is designed with a matching demultiplexing optical characteristic of the two sub-apertures in 106 so that imaging light beam from one sub-aperture is reflected to imaging device 112, while the imaging light beam from the other sub-aperture is transmitted to imaging device 114.

The imaging devices 112 and 114 are synchronized in their image capturing action so that pairs of images are generated at exactly the same instant, and their relationship is kept throughout the later stages of stereo image processing and display as a registered stereo pair. Due to the spatial separation of the two sub-apertures in 106, the pair of images would introduce stereoscopic vision to the observer when the images are displayed on a stereoscopic display device.

In one of the proposed multiplexing/demultiplexing designs, two linear polarizers with orthogonal polarizations are inserted into the two sub-apertures. A polarization beam splitter cube is used as 110, in which the polarization direction of the cube is aligned exactly with that of the polarizers inserted in the aperture stop 106.

The captured image pairs, in their digital form, are sent from the distal end through electric wires in cable 124 and 125 to the rest of the endoscope. The electric power supply and control commands for the electronic imaging sensor 112 and 114 are also sent to the sensors through wires in the cable 124. The benefits include the fact that the size of the electric cable 124 is much smaller than that of rigid relay lenses or optical image transmitting fiber bundles used in conventional designs, and the fact that the cable 124 will not limit the resolution of the endoscope. Due to the mechanical flexibility of the electric cable 124 and 125, the endoscope can be made with either flexible or rigid insertion tubes. Therefore, the platform technology proposed here can be extended to cover both rigid and flexible endoscopes. Once the optical images are converted into electronic signals, the flexible endoscope could be made with small diameter and very flexible insertion tube.

In terms of illumination optics, a solid state light source 126, made of either LEDs or laser diodes, with either broad or narrow spectral bandwidth, is placed behind the imaging sensors. The electrical power to the solid state light source 126 is supplied through the electric cable 128. Additional sensors or measuring devices, including thermal sensor and color sensor, can be implemented around the light source 126 to ensure its steady performance, and also to communicate with the micro controller in the base station of the endoscope system through the electric wires in cable 128. The emitted light from the light source 126 is projected into an optical homogenization device 118, which can be made of a light pipe, in order to generate uniform illumination in term of angular and spatial light distribution in brightness and color for the optical fibers 116 at the other end of the homogenizer 118. The homogenization device 118 is also designed to increase the optical coupling efficacy between the light source 126 and the optical fibers, 116. The optical fibers are spread around the lens cell 105 for the imaging optics, to form a very thin, but uniform illumination ring behind the illumination window 102 of the endoscope.

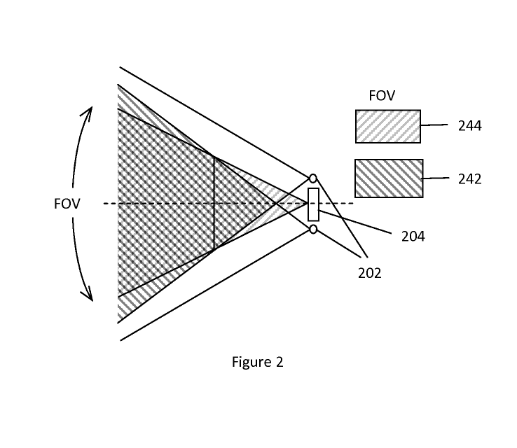

To produce images with uniform brightness, the optical illumination to the object must be both uniform and with certain unique distribution patterns. Meanwhile, in order to reduce haziness in images and increase image contrast, it is desirable to make the illumination field 242 slightly larger than, but still cover the field of view (FOV) 244 of the endoscope, as shown in FIG. 2. An optical illumination window component 102, which is designed to change the direction and angular distribution of the illumination light from the optical fiber ring 116, is arranged in the front of fiber ring end. The illumination window component 102 is made of a wedged transparent optical material with multi-facet surfaces on the side facing the fiber ring. The outside surface of the illumination window component 102 is preferably smooth and forms the optical window for the illumination light. The illumination window also seals the gap between the imaging optics and the shank of the endoscope.

In addition to the imaging and lighting consideration, there are also other issues that must be considered; and these include thermal dissipation, electrical grounding, the shielding from electromagnetic interference (EMI), data transfer rate and electrical insulation. For example, although a solid state light source is relatively highly efficient, it can still generate certain amount of heat and hence requires proper cooling design for a heat sink in order to maintain a desired working temperature range for the light source. As shown in FIG. 1, a heat sink device 130/132 made of materials of good heat conduction can be attached to the back of the solid state light source 126. The size of the heat sink 130/132 can vary depending on the efficiency of the light source and expected working time of the endoscope. The heat sink 130/132 can also be attached to a thin metal tube wall 122, which can in effect increase the size and mass of the heat sink. The heat sink 130/132 can also function as the electrical ground for the solid state light source 126. Properly grounded electrically, the thin metal wall tube 122 not only provides mechanical strength and protection for the precision opto-electronics in the imaging unit, but also offers much needed shielding for electromagnetic interference. Depending on the specific requirement, the thin metal wall tube 122 could also be replaced by a thin electrically conductive coating or an electro-plated surface deposited onto the inner surface of the outer tube 120, to provide needed shielding from electromagnetic interference (EMI).

The thin outer wall tube 120 is placed outside the metal tube 122 and can be made of an electrically insulating and biocompatible material. The tube 120 provides supplementary electrical isolation for the endoscope, which is required for medical devices in contact with patients' inner anatomy. In another design, tube 120 can also be removed and the metal tube 122 becomes the exterior wall of the endoscope, if the required electric insulation is sufficient by using well known basic insulation techniques.

If more stringent shielding from electromagnetic interference is required, one of the surfaces of the optical modules 104 and 108 could be coated with an electrically conductive but optically transparent layer such as an indium tin oxide (ITO) layer. When the surface is grounded electrically to the metal tube 122, a full enclosure (shielding) of the electronic components in the disposable unit of the endoscope is formed. Another option is to coat the surface of the aperture plate with electrically conductive but optically transparent layer, if an optically transparent substrate is used; or to make the opaque portion of the aperture from an electrically conductive material.

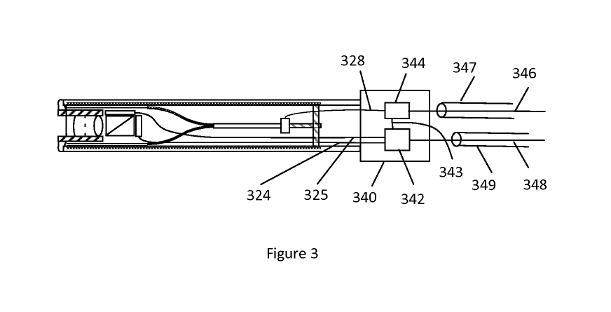

The working length of the electrical wires 124, 125 often is limited by the required data transmission rate of live images and I/O communication port. As a solution in the present invention, an electronic repeater is installed in the proximal portion of the endoscope. As shown in FIG. 3, a power conditioning/distribution module 344 is powered through an electric wire 346, which supplies DC power to solid state light source via a cable 328 and also to the imaging sensors via a cable 343 and an I/O conditioning module 342. The power module 344 also controls the functions of the solid state light source, and distributes power for I/O conditioning module 342. Module 342 is a two-way digital signal condition unit which converts signals from the imaging sensors through wires 324 and 325 to the preferred digital formats so that the signals could be transmitted to another receiver located far away by wires 348. At the same time, the electric signals from a distant location can be relayed to the module 342 through wires 348, and further to the imaging sensors after being converted into a format that can be recognized by various sensors. The cable 343 not only supplies power to module 342, but also relays control commands for the solid lighting source between module 342 and module 344. The returned performance monitoring data from the light source is then converted at the module 342 into ones with standard I/O protocols and is transmitted between module 342 and far away receivers. It is important to point out that although the term “convert” is used in the above discussion, the signal conditioning module 342 does not necessarily have to convert the data formats of the transmitted data, it could act as a two-way repeater to enhance the strength of the digital signals passing through it, so that the signal could be transmitted over long distance.

When properly grounded, the electrically conductive shielding 347 and 349 reduce the effect of electromagnetic emission from the wire 346 and 348, and also provide protection from potential EMI (Electromagnetic Interference) in operating environment.

In stereoscopic endoscopy, it is important to track the orientation of the stereoscopic endoscope during operation and adjust the orientation of displayed stereo images accordingly. In order to achieve orientation tracking, an electronic senor capable of detecting the orientation of the endoscope can be added into the module 342. The sensor is in constant communication with the image processor in the main system through cable 348.

A writable memory chip can also be added to the module 342. It will record the manufacturing, initial optical and electronic calibration information, and product ID, including the type of endoscope, illumination, stereo base settings, in the encrypted format. Once connected with a base station, the memory chip will transfer its recorded information to the system for keeping the information in a log book. The information helps to automate the initial setup process and ease the registration of the endoscopes during the operation. The information relating to the product ID at the base station and the condition of usage is also stored into the memory chip in module 342. More importantly the number or accumulated duration of usages for the disposable assembly is also recorded so that it could automatically cease to function when the preset limit is reached. Depending on specific applications, the medical endoscope may require either Basic Insulation with Protective Earth, or Double (Reinforced galvanic) Insulation to provide protection against the electric shock.

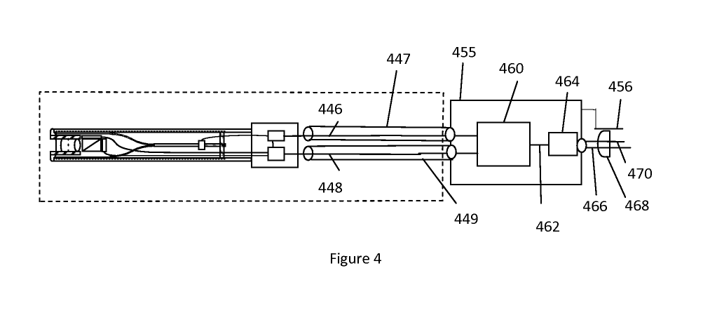

FIG. 4 shows the Basic Insulation design with Protective Earth. In this case the cable shieldings of 447 and 449 are directly connected to a metallic instrument enclosure 455 of the image processing unit 460 and a power conditioning unit 464. Meanwhile, a three-prong power cord 468 is used, in which the ground blade Protective Earth 456 is attached to any exposed conductive part, including instrument enclosure 455. The AC power cords 466 and 470 are attached to a power conditioning unit 464, which may include a medical grade isolation transformer, an uninterrupted power supply and DC power supplies. The power conditioning unit 464 provides basic insulation, a limit to leakage current and a regulated DC power for the whole system as desired to meet a satisfactory regulatory requirement. The DC power needed by the disposable unit is supplied through wire 446.

The digital data from the imaging sensors and other 1/0 communication are transmitted through wires 448 to the image processing/display unit 460. Inside the unit 460, the two channels of registered stereo video signal are processed separately and sent to a stereoscopic display device, from which a live stereoscopic video image of the object scene is displayed. The power for unit 460 is supplied through the power cord 462 from the power conditioning unit 464.

The endoscopic imaging system may be designed so that the endoscope itself is disposable. As shown in FIG. 4, the portion of system outlined by the dot lines could be attached and detached from the main system with proper electrical protection.

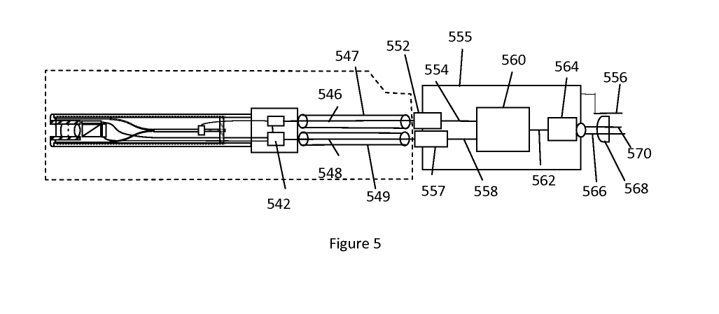

In the Double Insulation design, as shown in FIG. 5, a galvanic isolation device 557 is used to allow signals (data) to travel between the 1/0 conditioning module 542 and image processing unit 560 and meanwhile to prevent a direct flow of electric current between the module 542 and the unit 560. Preferably, certified opto-couplers are used for the reinforced isolation in module 557. A DC/DC converter 552 with high voltage isolation can also be used to prevent direct flow of DC current into the endoscope. The cable shielding 547 and 549 are isolated from the conductive enclosure 555 in proposed design.

The three-prong power cord 568 is used where the ground blade Protective Earth 556 is attached to any exposed conductive part, including instrument enclosure 555. The AC power cords 566 and 570 are attached to a power conditioning unit 564, which may include a medical grade isolation transformer, an uninterrupted power supply and DC power supplies. The power conditioning unit 564 provides basic insulation, set a limit to leakage current and also regulate DC power for the whole system so that the satisfactory regulatory requirement is met. The DC power needed by the disposable part of the endoscope is supplied through wire 554, then DC/DC converter 552 and wire 546.

The digital signals from the imaging sensors and other I/O ports of the disposable part of the endoscope are transmitted through wires 548, then galvanic isolator 557 and wire 558 to the image processing/display unit 560. Inside the unit 560, the two channels of registered stereo video signals are processed separately and sent to a stereoscopic display device, from which live stereo video of the endoscopic scene is displayed. The power for unit 560 is supplied through the power cord 562 from the power conditioning unit 564. The endoscopic imaging system may be designed so that endoscope itself is disposable.

As shown in FIG. 5, the portion of system outlined by the dotted lines could be attached and detached from the main system with proper electrical protection. The electric contacts in the disposable endoscope outlined in FIG. 5 will have Double insulation from the main power system.

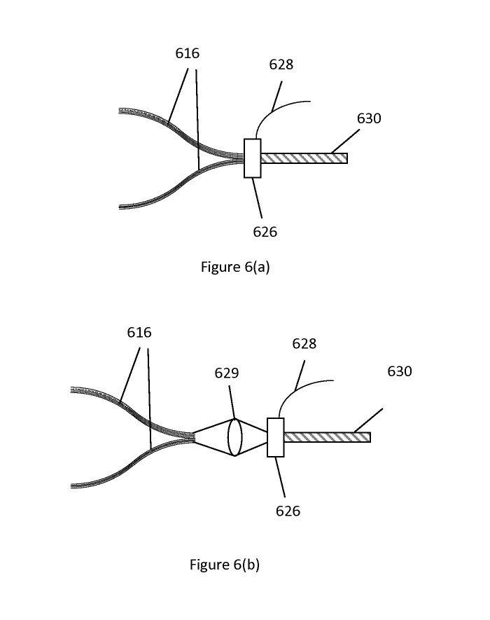

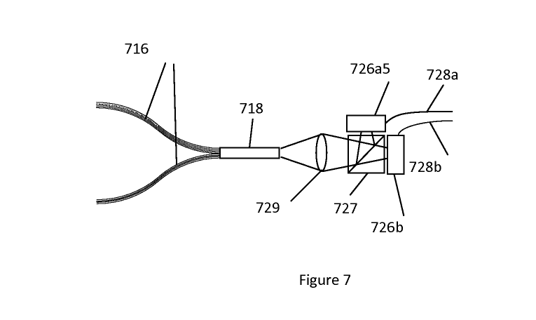

The proposed digital endoscope is designed to be illuminated by solid lighting source, including LEDs, laser diodes, superluminescent light emitting diodes (SLEDs), etc. Although a light homogenization device 118 is used in the design illustrated in FIG. 1, depending on the type of light source being used, the homogenizer 118 may not be needed. As shown in the FIG. 6, the illumination light from the solid light source 626 may be directly coupled into the fiber cable 616, where the light source 626 can consist of a single or multiple light emitters, with single or multiple wavelengths, or phosphor over emitter with single wavelength. FIG. 6 shows a direct coupling by either making a direct contact between fiber cable and light source (FIG. 6(a)) or using a condensing lens 629 (FIG. 6(b)). FIG. 7 illustrates another design, in which an optical beam combiner 727 is used to combine light beams from at least two light sources (726a and 726b) with different wavelengths, to form an equivalent light source of multiple wavelengths. The two light source 726a and 726b can both emit light with multiple but different wavelengths, or one can emit light with single narrow wavelength while the other with broad or multiple wavelengths. The light beam can be coupled into the fiber cable directly through a condensing optical lens 729, and/or into a light homogenization device 718 and then be projected into the fiber cable 716 for better performance.

The solid state light source in the proposed endoscope can be driven in the form of pulse width modulation (PWM) with a relatively high repetition rate and constant peak electric current, in order to maintain stable color output. The apparent intensity of the light source can be adjusted by changing the duty cycle of the pulse modulation. Since the repetition rate is much higher than what a human eye could perceive, the light from the light source appears continuous. The light output from the solid state light source could also be further switched at the frame rate of the imaging sensor and turned on only when the shutters for the two imaging sensors are open, behaving like a constant strobe light.

By shortening the duration of light pulse and increasing the pulsed light intensity, the strobe light helps reduce the blurring of the images caused by the motion of the endoscope.

What is claimed is:

1. A disposable simultaneous stereo endoscope system, comprising,
an imaging sub-system having two sub-apertures for defining the two stereo imaging channels and a demultiplexing beam splitter for splitting the two light beams to two imaging devices,
an illumination sub-system further comprising a solid light source, an optical fibers based ring illumination path, and a wedged multi-facet illumination window,
an electrically conductive heat sink for dissipating the heat generated by the solid light source and also for shielding end side of the endoscope,
a metallic tube for shielding the opto-electronic components inside the endoscope from electromagnetic interferences,

2. The endoscope of claim 1, wherein said illumination sub-system further comprises a light homogenization device for homogenizing the illumination light from the solid light source to the optical fibers.

3. The endoscope of claim 1, wherein said illumination sub-system further comprises an optical beam combiner for combining illumination light beams from at least two light sources of different wavelengths.

4. The endoscope of claim 1, wherein said illumination sub-system further comprises condensing optical lens for focusing the illumination light from the solid light source into the optical fibers.

5. The endoscope system of claim 1, further comprising an outer tube made from an electrically insulating and biocompatible material.

6. The endoscope system of claim 1, further comprising means for coating or depositing an optically transparent and electrically conductive layer on at least one of the surfaces of the imaging optics to achieve a complete electric shielding for the disposable endoscope.

7. The endoscope of claim 1, wherein said illumination sub-system creates an illumination field that is larger than the field of view of the imaging sub-system for the desired imaging space of the endoscope.

8. The endoscope of claim 1, further comprising means for electrically connecting the disposable imaging and lighting endoscope with a repeater unit containing a power conditioning/distribution module and an input/output conditioning module to increase the data transfer rate over a relatively long distance.

9. The endoscope of claim 1, further comprising an image processing and power conditioning unit and an electrical isolation means to provide protection against electric shock.

10. The endoscope of claim 9, wherein said electrical isolation means comprises a basic insulation design in which electrical cable shielding is directly connected to the metallic instrument enclosure of the image processing and power conditioning unit.

11. The endoscope of claim 9, wherein said electrical isolation means comprises a double insulation design in which a galvanic isolation device is used to allow signals to travel through and meanwhile to prevent a direct flow of electric current.

12. The endoscope of claim 1, further comprising an orientation detection sensor for sensing the orientation direction of the endoscope so that the displayed stereo video images can be adjusted based on the orientation of the endoscope.

13. The endoscope of claim 1, further comprising writable memory chip for recording the information about the disposable device and its usage.

14. The endoscope of claim 1, wherein said solid light source is driven in the form of pulse width modulation (PWM) with constant peak electric current to maintain stable color output and a high enough repetition rate so that the illumination light appears continuous to a human eye.

15. The endoscope of claim 14, wherein said pulse modulation is further turned on only when the shutters for the two imaging sensors are open

16. The endoscope of claim 14 wherein said pulse modulation has a small duty cycle and a high peak light intensity for reducing the blurring of the images caused by the motion of the endoscope.
